(19) United States
(12) Patent Application Publication
(10) Pub. No.: US 2008/0102051 A1
(43) Pub. Date: May 1, 2008

(54) Use of polyols to increase stiffness in low voc hair styling products

(75) Inventors: Angelika F. Huynh, Walnut, CA (US); Karen Hohenstein, Irvine, CA (US); Mary Rose Santos, Placentia, CA (US)

Correspondence Address: THE DIAL CORPORATION, 15501 N. DIAL BOULEVARD, SCOTTSDALE, AZ, 85260, US

(73) Assignee: Henkel KGaA

(21) Appl. No.: 11/980,309

(22) Filed: Oct. 30, 2007

Related U.S. Application Data
(60) Provisional application No. 60/855,530, filed on Oct. 31, 2006.

(51) Int. Cl. A61K 35/24; A61K 36/00; A61K 8/73; A61K 8/81; A61P 43/00

(52) U.S. Cl. 424/70.13; 424/537; 424/70.15; 424/70.16; 424/725

(57) ABSTRACT

Low to no-VOC hair styling compositions are described that comprise at least one synthetic polymer or copolymer, a starch, modified starch or other cellulosic material and a polyol. Due to the presence of at least one polyol, the compositions of the present invention provide hard-hold to hair with great durability, flexibility and minimal flaking.

### PRIORITY APPLICATION

The present application claims priority to U.S. Provisional Application 60/855,530 filed Oct. 31, 2006 and entitled “USE OF POLYOLS TO INCREASE STIFFNESS IN LOW VOC HAIR STYLING PRODUCTS”, which is incorporated herein.

### FIELD OF INVENTION

The present invention relates to hair hold compositions and specifically to aqueous hair styling compositions that include low- to no-volatile organic compounds yet still provide hard-hold to hair due to the incorporation of one or more polyols in the polymeric composition.

### BACKGROUND

It is very desirable in personal grooming to maintain one's hair in a particular setting, and a common method of accomplishing this is to apply a hair styling composition to the hair and allowing it to dry and set either on its own or with heat from a hair dryer or hot curlers. Hair setting compositions can assist in styling the hair and they provide a temporary hold to the hair while imparting shine and appearance benefits as well. Hair styling products may be marketed in various forms, including hair spray, styling gel, setting foam or hair lacquer. The objective in formulating hair styling gels has always been to optimize a number of interrelated physical properties of the product seen both before and after drying. These important parameters include a desirable viscosity for the product such that it dispenses nicely (when squeezed from a plastic tube for example) and feels acceptable in the hands and hair when applied to the hair. Also desirable is to balance the final level of hold when dry with the ability to wash out again with shampoo, and to minimize flaking. These parameters are evaluated by various visual and tactile sensory methods including viewing, touching, combing/brushing, looking at shine, cleanliness, stiffness, tackiness, bounce, ability to re-style, combing ease, residue/flaking, static, smoothness and the like.

Conventional compositions for hair styling products are well known in the art. Most commercial hair styling compositions include hair setting polymers (styling or fixative), natural gums and resins, viscosifier polymers, polymer modifiers, solvents, co-solvents, colors, fragrances, preservatives and the like. Styling and fixative polymers typically include nonionic and anionic fixative polymers, also known as film former polymers. Among the most commonly used styling and fixative polymers and copolymers are mixtures of vinyl acetate and vinylpyrrolidone along with anionic polymers or combinations of anionic polymers with nonionic or cationic polymers. Some commercially available polymers that have been used for some time now in various combinations include polyvinylpyrrolidone (PVP), sold under the trade name of Luviskol-PVP® from BASF, polyvinylpyrrolidone/vinylacetate copolymer (PVP/VA), sold under the trade name Luviskol-VA® from BASF, vinyl pyrrolidone/dimethylaminoethyl methacrylate copolymer, sold under the trade name of Styleze® from ISP, vinyl caprolactam/vinyl pyrrolidone/dimethylaminoethyl methacrylate copolymer with 0.77% ethanol, sold under the trade name of Advantage®-S Solution from ISP, and vinyl caprolactam/vinyl pyrrolidone/dimethylaminoethyl methacrylate copolymer with 63% ethanol, sold under the trade name of Advantage® LC-A from ISP.

When hair setting resins and fixative polymers alone cannot provide the desired viscosity for the hair setting product, formulators may add rheology modifiers or gellants. These ingredients may include polymers, gums or resins, or other rheology modifiers such as emulsifiers, waxes, and the like, to achieve the target rheology for the product.

When a hair setting composition is dried on the hair, it is important to optimize strength and elasticity of the film on the hair, and to ensure the dried film on each hair shaft does not flake or dust when the hair is subject to stress throughout the day, such as combing. Additionally, the set hair should be tack-free, clear, glossy, humidity-resistant, yet still be able to be easily washed out of the hair with shampoo products. To achieve these desirable attributes, it is common to blend both synthetic copolymers of the type mentioned above with carbohydrate or starch-based materials such as hydroxyethylcellulose and/or modified corn starch, the latter sold under the trade name Amaze® from National Starch. Such combinations of starches and their derivatives with synthetic vinyl copolymers are thoroughly mentioned in U.S. Pat. No. 6,413,505 issued to Vitale et al. and incorporated herein by reference. Other ingredients that help optimize the physical properties of hair gel include emulsifiers, petrolatum, and various fatty alcohols.

Most hard-hold hair styling products on the market today rely on high concentrations of polymers and copolymers to provide a desirable stiff feel and high level of hold. Many polymers are typically supplied as ethanolic solutions so that the end formulator can use a fairly concentrated raw material that still can be poured into the mixer. New regulatory initiatives to improve air quality have centered on reduction of VOC's in many household and industrial products, and these regulations have affected the formulation of personal hair care products. For example, new regulations in the state of California will eventually limit VOC level in hair care holding gels to 2% maximum. If a formulator simply substitutes aqueous polymer solutions for the ethanolic solutions, the resulting compositions impart less stiffness in the hair and the hold is dramatically reduced. Formulators have tried to compensate for this lack of hold by simply increasing the amount of the polymers. However, simply increasing the amounts of various polymers in an aqueous formula will not improve the formula's hard-hold characteristics. Such a strategy merely increases the cost of the formulation and increases brittleness and flaking of the dried composition in the hair. Consequently it is desirable to formulate low- to no-VOC hair gels that have an absence of ethanolic solutions of raw materials, but still have consumer acceptable characteristics. More particularly, what is needed in the industry and what is entirely lacking in the art today is a way to formulate low- to no-VOC hair styling compositions that still provide consumer acceptable feel, hold and shine without undue flaking.

### SUMMARY OF THE INVENTION

The present invention provides low to no-VOC hair styling and setting compositions that have remarkable hard-hold characteristics when dried on the hair. These compositions have little to no-VOC, yet still impart hard-hold to hair with little flaking, and are made possible by incorporation of polyols. Not being bound by any theory, the polyols appear to strengthen the film formed on the shafts of the hair, possibly through crosslinking of the polymers present in the compositions or by operating as plasticizers affecting crystallinity.

The compositions of the present invention have less than 2% VOC content and minimally comprise; (a) at least one synthetic polymer or copolymer; (b) at least one starch or other cellulosic material; (c) water; and, (d) at least one polyol. The polyols that find use in the present invention include the straight chain small organic molecules, cyclic and cyclic/straight chain carbohydrates with as many as 12 carbons, some of which are known as sugar alcohols and used in the food industry.

### DETAILED DESCRIPTION OF THE INVENTION

The following description is of exemplary embodiments only and is not intended to limit the scope, applicability or configuration of the invention in any way. Rather, the following description provides a convenient roadmap for implementing exemplary embodiments of the invention. Various changes to the described embodiments may be made in the function and relative amounts of the ingredients described without departing from the scope of the invention as set forth. Additionally, though demonstrated herein in terms of water-based, non-aerosol hair setting gel compositions with relatively high viscosity, other product forms the present invention may take include hair sprays, pourable liquids, sprays, spritzes, foams, cremes, pastes, non-runny gels, mousses, pomades, lacquers, non-aerosol pump-spray liquids and the like. Furthermore, the compositions of the present invention may include adjuvants such as deodorants, fragrances, aromatherapy essences, herbs, infusions, antimicrobials, pediculicides, stimulants, essential oils, hair coloring, dying or tinting agents, anti-gray agents, sun-blockers, vitamins, antioxidants, surfactants and other wetting agents, Rogaine®, mica, silica, metal flakes or other glitter-effect materials, conditioning agents, anti-static agents, opacifiers, detackifying agents, penetrants, preservatives, and emollients and the like, and these compositions having additional consumer benefits (perceivable or otherwise) likewise fall within the ambit of the present invention.

That being said, the present invention provides compositions useful for styling and setting hair that comprise polymers, copolymers, starches and most importantly, polyols. Most remarkable, these compositions are low- to no-VOC yet still impart hard-hold to hair when dried on the hair, and this is possible by incorporating various polyols that improve the hardness and durability of the resulting film on the hair. The compositions of the present invention minimally comprise;
(a) at least one synthetic polymer or copolymer;
(b) at least one starch or other cellulosic material, natural or modified;
(c) water; and,
(d) at least one polyol, wherein the final composition contains less than 2.0% volatile organic compounds (VOC).

Additionally, the compositions of the present invention may include viscosity modifying polymers including cellulosic materials (for example for thickening), an oil phase comprising petrolatum or the like, fatty alcohols, emulsifiers, and various oil-soluble film formers, optional solvents and co-solvents selected from the group considered “VOC-exempt” (acetone, d-limonene and the like), and any combination of the adjuvants mentioned previously to either impart marketable benefits or to adapt the basic formulations comprising the essential ingredients (a)-(d) to a particular physical product form and delivery method/preferred packaging, (liquid, gel, pump, aerosol, etc.).

Multiplying each of the solvent levels present in the individual raw materials by the amount of that raw material used in the composition, and then adding these fractional amounts, give the “VOC content” or “percent volatile organic compounds” in a composition. Ethanol, for example, may be 100% VOC contributing raw material (when absolute alcohol), whereas some raw materials, such as water, various powdered polymers and starches, or water based solutions of polymers, may contribute no VOC at all to the composition. Frequently however, synthetic polymers are obtained in alcoholic solution (for example, 20-80% solvent) and using these ingredients adds to the VOC content of the final composition.

Polyols of use in the present invention include but are not limited to straight-chain molecules having carbon chain length of from about 3 to about 8, most preferably from about 5 to about 6, with the number of hydroxyl groups from 3 to about 8, most preferably from about 5 to about 6. “Polyols” in the content of the present invention are understood to be substances with 3 or more hydroxyl groups (that is, including triols but excluding diols). For example, of use in the present invention are straight chain polyols selected from the group consisting of erythritol, glycerol, mannitol, sorbitol and xylitol. The polyols that find use in the present invention may be natural or synthetically derived, and may also include cyclic, or combinations of straight chain and cyclic structures. For example, isomalt, lacitol, maltitol, and various HSH's (hydrogenated starch hydrosylates) may find use in the present invention. Out of all the sugar alcohols and simple polyols, most preferable is to use mannitol or sorbitol (both C₆H₁₄O₆) or combinations of the two. Polyols that contain, in addition to the requisite minimum (3) hydroxyl groups, other functional groups in the molecule such as aldehydes, ketones, carboxylate, thiols, etc., are also of use in the present invention. For example, methyl 2,5,6-trihydroxyhexanoate, or other functionalized polyols may find use herein. Additionally, natural sugar monosaccharides that are in equilibrium mixture of open straight chain form and cyclic aldol or ketol form may also find use in the present invention. Many small organic molecules, or mixtures of them, may find use in the compositions of the present invention for helping to provide hard-hold provided they minimally have three or more hydroxyl groups. The polyol or polyols is/are preferably incorporated in the compositions of the present invention at from about 5% to about 15% by weight in the composition. Preferred also is to maintain a ratio of polyol to total synthetic and natural polymers of from about 1:3 to about 1:10.

The polymer or copolymer that forms an essential component of the compositions of the present invention may be selected from the group consisting of nonionic, anionic, zwitterionic or amphoteric or cationic synthetic homopolymers, and copolymers. For example, nonionic polymers of use in the present invention include polyvinylpyrrolidone, copolymers of N-vinylpyrrolidone and vinyl acetate, and/or vinyl propionate, polyvinylcaprolactam, polyvinylamides and salts thereof, and copolymers of vinylpyrrolidone and dimethylaminoethyl methacrylate, terpolymers of vinylcaprolactam, vinylpyrrolidone and dimethylaminoethyl methacrylate, polysiloxanes and the like.

Anionic polymers of use in the present invention include vinyl acetate/crotonic acid, vinyl acetate/acrylate and/or vinyl acetate/vinyl neodecanoate/crotonic acid copolymers, sodium acrylate/vinyl alcohol copolymers, sodium polystyrenesulfonate, ethyl acrylate/-N-tert-butylacrylamide/acrylic acid copolymers, vinylpyrrolidone/vinylacetate/itaconic acid copolymers, acrylic acid/acrylamide copolymers and/or sodium salts thereof, homo- and/or copolymers of methacrylic acid and/or salts thereof, and acrylate/hydroxyacrylate, octylacrylamide/acrylate or methacrylate and/or butyl acrylate/N-vinylpyrrolidone copolymers or polystyrenesulfonates.

Amphoteric polymers that find use in the present invention include copolymers of N-octylacrylamide, methacrylic acid and tert-butylaminoethyl methacrylate of the “amphomer” type, copolymers of methacryloylethylbetaine an alkylmethacrylates of the “yukaformer” type, copolymers of monomers containing carboxyl groups or sulfone groups, for example methacrylic acid and itaconic acid, with basic group-containing monomers such as mono- or dialkylaminoalkyl methacrylates and/or mono- and dialkylaminoalkyl methacrylamides, copolymers of N-octylacrylamide, methyl methacrylate, hydroxypropyl methacylate, N-tert-butylaminoethyl methacrylate and acrylic acid.

Cationic polymers that may find use in the present invention include vinylpyrrolidone/vinylimidazolium methochloride copolymers, quaternized vinylpyrrolidone/diakylaminoalkyl methacrylate copolymers, cationic cellulose derivatives, such as hydroxyethylcellulose/dimethylalkylammonium chloride copolymers, and terpolymers of vinylcaprolactam/vinylpyrrolidone with dimethylaminoethyl methacrylate or vinylimidazolium methochloride and acrylamido copolymers.

That being said, the preferred polymers for use in the present invention include but are certainly not limited to vinyl pyrrolidone/dimethylaminoethyl methacrylates copolymer, vinyl acetate/crotonates/vinyl neodecanoate copolymer, octyl acrylamide/acrylates/butylaminoethyl methacrylate copolymer, vinyl acetate/crotonates, polyvinylpyrrolidone (PVP), polyvinylpyrrolidone/vinyl acetate copolymer, PVP acrylates copolymer, vinyl acetate/crotonic acid/vinyl proprionate, acrylates/acrylamide, acrylates/octylacrylamide, acrylates copolymer, acrylates/hydroxyacrylates copolymer, and alkyl esters of polyvinylmethylether/maleic anhydride, diglycol/cyclohexanedimethanol/isophthalates/sulfoisophthalates copolymer, vinyl acetate/butyl maleate and isobornyl acrylate copolymer, vinylcaprolactam/PVP/dimethylaminoethyl methacrylate, vinyl acetate/alkylmaleate half ester/N-substituted acrylamide terpolymers, vinyl caprolactam/vinylpyrrolidone/dimethylaminoethyl methacrylates copolymer, vinyl caprolactam/vinylpyrrolidone/methacrylamidopropyl trimethylammonium chloride terpolymer, methacrylates/acrylates copolymer/amine salt, polyvinylcaprolactam, polyurethanes, polyquaternium-4, polyquaternium-10, polyquaternium-11, polyquaternium-46, hydroxypropyl guar, hydroxypropyl guar hydroxypropyl trimmonium chloride, polyvinyl formamide, polyquaternium-7, and hydroxypropyl trimmonium chloride guar.

The most preferred polymers for use in the compositions of the present composition are various ratios and amounts of polyvinylpyrrolidone, polyvinylpyrrolidone/vinyl acetate copolymer, vinyl pyrrolidone/dimethylaminoethyl methacrylates copolymer and vinyl caprolactam/vinyl pyrrolidone/dimethylaminoethyl methacrylates copolymer. The preferred total amount of synthetic fixative polymers is from about 30% to about 70%, with either one of these or combinations thereof. In order to keep the VOC level in the composition at or below 2%, it is important to use polymers that are available in aqueous or nearly aqueous solution, or in powdered form, rather than using polymers supplied in solvent solutions that will contribute to the VOC content of the final composition. That being said, the most preferred polymers for use in the present compositions include polyvinylpyrrolidone 20% aqueous solution, or polyvinylpyrrolidone powder, sold under the trade names of Luviskol K-90® and Luviskol-PVP® respectively from BASF (both 0% VOC contribution), polyvinylpyrrolidone/vinylacetate copolymer (PVP/VA-copolymer), sold under the trade name Luviskol VA-73W® from BASF (0% VOC contribution), vinyl pyrrolidone/dimethylaminoethyl methacrylate copolymer (VP/DMAPA-copolymer), sold under the trade name of Styleze CC-10® from ISP (0% VOC contribution), vinyl caprolactam/vinyl pyrrolidone/dimethylaminoethyl methacrylate copolymer with 0.77% ethanol, sold under the trade name of Advantage®-S Solution from ISP (0.77% VOC contribution), and (less favorably) vinyl caprolactom/vinyl pyrrolidone/dimethylaminoethyl methacrylate copolymer with 63% ethanol, sold under the trade name of Advantage® LC-A from ISP (63% VOC contribution).

The compositions of the present invention also include starch, modified starches and/or other cellulosic material or combinations of these, for modifying the quality of the dried film on the hair and for modifying the viscosity of the actual composition. For example, of use in the compositions of the present invention include celluloses, cellulose derivatives, cellulose gums, ethoxylated celluloses, starch or gums, guar gum, guar hydroxypropyl trimonium chloride, xanthan gum, hydroxypropyl guar, karaya gum, and the like. Also of use in the present invention are pregelatinized crosslinked starch derivatives, including hydroxypropyl distarch phosphate, as described in U.S. Patent Application Publication US2005/0191264 and incorporated herein by reference. Preferred cellulosic materials of use in the compositions of the present invention include nonionic or cationic saccharides such as cellulose ethers including methyl cellulose, carboxymethyl cellulose, hydroxy propyl methyl cellulose, hydroxyethyl cellulose, hydroxypropyl cellulose, and ethyl hydroxyethyl cellulose, dextrans obtained from Sigma, Kitamer PC, a chitosan carboxylate and Kytamer L, a chitosan lactate obtained from Amerchol, Gafquat HS-100, Polyquaternium-28 from International Specialties, polyquaternium4, polyquaternium-10, sodium alginate, agarose, amylopectins, amyloses, arabinans, arabinogalactans, arabinoxylans, carrageenans, gum arabic, cellulose derivatives such as methylcellulose, hydroxypropylmethylcellulose, hydroxyethyl cellulose, carboxymethylcellulose, carboxymethylguar gum, carboxymethyl(hydroxypropyl)guar gum, hydroxyethylguar gum, hydroxypropylguar gum, cationic guar gum, chondroitins, chitins, chitosans, cocodimonium hydroxypropyl oxyethyl cellulose, colominic acid [poly(N-acetyl-neuraminic acid], corn starch, curdlan, dermatin sulfate, furcellarans, dextrans, cross-linked dextrans known as dextranomer (Debrisan), dextrin, emulsan, flaxseed saccharide (acidic), galactoglucomannans, galactomannans, glucomannans, glycogens, guar gum, or hydroxyethylstarch, hydroxypropylstarch, hydroxypropylated guar gums, gellan gum, glucomannans, gellan, gum ghatti, gum karaya, gum tragacanth (tragacanthin), heparin, hyaluronic acid, inulin, keratan sulfate, konjac mannan, laminarans, laurdimonium hydroxypropyl oxyethyl cellulose, liposan, locust bean gum, mannans, nigeran, nonoxylnyl hydroxyethyl cellulose, okra gum, oxidized starch, pectic acids, pectins, polydextrose, potato starch, protopectins, psyllium seed gum, pullulan, sodium hyaluronate, steardimonium hydroxyethyl cellulose, raffinose, rhamsan, tapioca starch, welan, levan, scleroglucan, stachyose, succinoglycan, wheat starch, xanthan gum, xylans, xyloglucans, and mixtures thereof. Microbial saccharides can be found in the fourth edition of Kirk-Othmer Encyclopedia of Chemical Technology, Fourth Edition. Vol. 16, John Wiley and Sons, NY pp. 578-611, 1994. Complex carbohydrates can be found in the fourth edition of Kirk-Othmer Encyclopedia of Chemical Technology, Fourth Edition. Vol. 4, John Wiley and Sons, NY pp. 930-948, 1994. Of most particular use in the present invention is hydroxyethylcellulose and a modified corn starch from National Starch found under the trade name Amaze®. Most preferred for use in the compositions of the present invention is from about 0.01% to about 10% total cellulosic and starch materials, by weight in the composition. Most preferred is to use hydroxyethylcellulose and Amaze® modified corn starch from National Starch, together in the composition at a total of from about 0.1% to about 5% by weight.

The compositions of the present invention may also include a thickening polymer, such as a high molecular weight polyacrylate. Thickeners can be utilized alone or in combination so long as the chosen thickeners are compatible with the hair styling composition. Thickeners may include, but are not limited to, acrylic acid homopolymers under the Carbopol® trade name from BF Goodrich, acrylates/C10-30 alkyl acrylate crosspolymer (Carbopol® 1342, 1382, Pemulins® TR-1 and TR-2 from BF Goodrich), Acrylates/Steareth-20 Itaconate Copolymer, Acrylates/Ceteth-20 Itaconate Copolymer from National Starch, Bentonite, PVM/MA Decadiene Crosspolymer from International Specialties Products, Acrylates/steareth-20 methacrylate copolymer, Acrysol® ICS-1 from Rohm and Haas Co., acrylamide/sodium acrylate copolymer, Hostacerin® PN 73, Hoecsht AG., acrylate copolymer (Antil® 208) supplied by Degussa-Goldschmidt, acrylic acid/acrylonitrogens copolymer (Hypan® SA-100H, SR-150H) supplied by Lipo, Acrylic/acrylate copolymer (Carboset® 514, 515, 525, XL-19, XL-19×2, XI-28, XL40, 526) supplied by BF Goodrich, Ammonium acrylateslacrylonitrogens copolymer (Hypan® SS-201) from Lipo, Quaternium-18 Bentonite, Sodium salt of crosslinked poly(acrylic acid) under the tradenames PNC® 430, PNC® 410, PNC® 400 from 3V, Stearalkonium Bentonite, Claytone, supplied by Southern Clay, Quaternium-18 Hectorite (Bentone 38), Stearalkonium Hectorite (Bentone 27) supplied by Rheox, acrylamide/sodium acrylate copolymer (Hostacerin PN 73) supplied by Hoechst, Poly(acrylic acid) known as Carbopol® 400 series (BF Goodrich) or Aquatreat® (Alco 3V), polyquaternium-18 (Mirapol® AZ-1) from Rhone Poulenc, polyquaternium-27, polyquaternium-31, polyquaternium-37, trihydroxystearin (Thixcin from Rheox; Flowtone from Southern Clay), Dimethylaminoethyl methacrylamide and acrylamide copolymer (Salcare® SC63 from Ciba Specialties), Acrylic polymer cationic thickening agents (Synthalen® CR and its related compounds) from 3V Sigma. Other thickeners and polymers can be found in the “The Encyclopedia of Polymers and Thickeners for Cosmetics,” Cosmetics and Toiletries, Lochhead, R., pp. 95-138, Vol. 108, (May 1993). Thickeners, if required in the composition, may be incorporated at from about 0.01% to bout 5% by weight active polymer.

The compositions of the present invention may also include petrolatum or other waxes and oils. Petrolatum is a mixture of hydrocarbons that finds use in various personal care products. Preferred petrolatum includes white petrolatum USP, petrolatum USP, mineral jelly and ointment base. The melting point ranges of the preferred petrolatum for use in the present invention may be from about 80° F. to about 135° F. Most preferred is to use UltraPure® Liquid Petrolatum USP from Ultra Chemicals that has a melting point range of about 105°-115° F., or various grades (ranging in color) of Penreco® Petrolatum USP having melting point ranges around 122°-135° F. The compositions of the present invention may also contain an emulsifying wax and/or oil. Such materials include the non-limiting examples of bees' wax, candelilla wax, carnauba wax, emulsifying wax (for example Polawax® from Croda) and Jojoba, safflower, canola (tribehenin), tallow, lard, palm, castor, sunflower seed, or soya bean oil oils, or hydrogenated derivatives thereof. Most preferred is to incorporate Polawax®, jojoba oil, safflower oil, tribehenin, and/or hydrogenated castor oil, singularly or in any combination. When desired in the present compositions, any combination of these materials may be used at from about 0.1% to about 5% by weight of the total composition. Most preferred is to use (individually or in combination) petrolatum, wax and/or oil at from about 1% to about 3% by weight in the composition.

Fatty alcohols that may find use in the compositions of the present invention include naturally derived and synthetic materials. These are high molecular weight straight or branched chain primary alcohols. Most preferred for use in the present invention include lauryl (C12), myristyl (C14), cetyl or palmityl (C16), stearyl (C18), oleyl (C18-unsaturated) and linoleyl (C18-polyunsaturated) alcohols, or combinations thereof. Ceteryl alcohol is also preferred and is a mixture of cetyl and stearyl alcohols. The fatty alcohol in the present invention may be incorporated at from about 0.01% to about 5% by weight in the composition.

Also of use as an optional ingredient in the compositions of the present invention include emulsifiers. Emulsifiers for use in cosmetic applications are amply listed in McCutcheon's Emulsifiers and Detergents. Many suitable emulsifiers are nonionic esters or ethers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditol as hydrophilic moiety. The hydrophilic moiety can contain polyoxypropylene. The emulsifiers additionally contain a hydrophobic alkyl, alkenyl or arylalkyl moiety, normally containing from about 8 to 50 carbons. The hydrophobic moiety can be either linear or branched and is often saturated, though it can be unsaturated, and is optionally fluorinated. The hydrophobic moiety can comprise a mixture of chain lengths, for example those deriving from tallow, lard, palm oil, sunflower seed oil or soya bean oil. Such nonionic surfactants can also be derived from a polyhydroxy compound such as glycerol or sorbitol or other alditols. Examples of such emulsifiers include ceteareth-10 to-25, ceteth-10-25, steareth-10-25 (i.e. C₁₆ to C₁₈ alcohols ethoxylated with 10 to 25 ethylene oxide residues) and PEG-15-25 stearate or distearate. Other suitable examples include C₁₀-C₂₀ fatty acid mono, di- or tri-glycerides. Further examples include C₁₈-C₂₂ fatty alcohol ethers of polyethylene oxides (8- to 12-EO). Other examples of useful emulsifiers are fatty acid mono or possibly diesters of polyhydric alcohols such as glycerol, sorbitol, erythritol or trimethylolpropane. The fatty acyl moiety is often from C₁₄ to C₂₂ and is saturated in many instances, including cetyl, stearyl, arachidyl and behenyl. Examples include monoglycerides of palmitic or stearic acid, sorbitol mono or diesters of myristic, palmitic or stearic acid, and trimethylolpropane monoesters of stearic acid. Another usable class of emulsifiers comprises dimethicone copolymers, namely polyoxyalkylene modified dimethylpolysiloxanes. The polyoxyalkylene group is often a polyoxyethylene (POE) or polyoxypropylene (POP) or a copolymer of POE and POP. The copolymers often terminate in C₁ to C₁₂ alkyl groups. Such suitable emulsifiers and co-emulsifiers are widely available under many trade names and designations including Abil®, Arlacel®, Brij®, Cremophor®, Dehydrol®, Dehymuls®, Emerest®, Lameform®, Pluronic®, Prisorine®, Quest PGPR®, Span® Tween®, SF1228, DC3225C and Q2-5200.

Preferred emulsifiers include any combination of fatty alcohols (such as mentioned previously), phosphate-based emulsifying waxes, sorbitan monooleates and stearates and other carbohydrate esters of fatty alcohols and their ethoxylated derivatives, and the polyalkylene glycols and polyethoxylated waxes. For example, a preferred fatty alcohol blend for optional use in the present invention is Crodafos® CES (white solid or flakes) from Croda, which is a blend of ceteryl alcohol, dicetyl phosphate and ceteth-10 phosphate. Of particular use in the present invention is the Apifil PEG-8 beeswax emulsifier, which is a combination of fatty acid esters and polyethylene glycol and is a nonionic self-emulsifying base. When desired in the composition, the preferred amount of the emulsifier is from about 0.01% to about 10% by weight in the composition.

Also, the compositions of the present invention may include “oil-soluble film former” polymers. Non-limiting examples of the oil-soluble film former include polymethylsilsesquioxanes; acrylic fluorinated emulsion film formers, such as Foraperle® film formers (e.g., Foraperle® 303 D available from Elf Atochem); GANEX® copolymers, such as butylated PVP, PVP/Hexadecene copolymers, PVP/Eicosene copolymers, and tricontanyl; Poly-(vinylpyrrolidone/diethylaminoethyl methacrylate) copolymers and PVP/Dimethylaminoethylmethacrylate copolymers such as Copolymer 845 available from I.S.P.; Resin ACO-5014 (Imidized IB/MA copolymer); other PVP based polymers and copolymers; silicone gums; cyclomethicone copolymers and dimethicone crosspolymers, such as Dow Corning® 2-9040 and those disclosed in U.S. Pat. No. 5,654,362, the disclosure of which is hereby incorporated by reference; trimethyl siloxysilicates such as SR 1000, 554230, and SS4267 available from GE Silicones; alkyl cycloalkylacrylate copolymers, such as those disclosed in WO 98/42298, the disclosure of which is hereby incorporated by reference; Mexomere® film formers and other allyl stearate/vinyl acetate copolymers; polyolprepolymers, such as PPG-12/SMDI copolymer, also called Poly-(oxy-1,2-ethanediyl), α-hydro-ω-hydroxy-polymer with 1,1′-methylene-bis-(4-isocyanatocyclohexane) available from Barnet; and Avalure® AC Polymers (Acrylates Copolymer) and Avalure® UR polymers (Polyurethane Dispersions), available from BFGoodrich. Preferred for use in the present invention is the already mentioned tricontanyl PVP copolymer sold under the trade name Ganex® WWP-660 from ISP. Any combination of the Ganex® V/WP grades are excellent film formers for the present invention and provide water and wear resistance and a moisture barrier to the set hair. The oil-soluble film former polymer may be present in the composition from about 0.1% to about 5% by weight.

Also optional to the compositions of the present invention is the addition of a chelant. Chelants that may find use herein include but are not limited to phosphates (organic and inorganic), NTA, the various ethylenediaminetetraacetic acid (EDTA) derivatives, and lower molecular weight polyacrylates. For example, the present invention may include trisodium or tetrasodium EDTA, various salts of NTA, phosphate esters, or Acusol® 445 from Rohm and Haas. The chelant may be present in the composition from about 0.001% to about 1% by weight.

Although the present invention relates to low- to no-VOC polymeric compositions for hair styling, there may be a need to add some solvent or co-solvent to the compositions of the present invention, for example to adjust physical parameters/consumer benefits or to adapt the base formulation (polymers, water, polyol composition) to a specific product form (liquid, gel, pump, aerosol, etc.). For example, it may be necessary to add small amounts (e.g., less than 1% or so) of VOC contributing solvents to the composition to help optimize the viscosity, stability, feel, dry time, etc., to the product. In addition to adding water, the primary solvent for the present invention, one may add alkanol solvents such as ethanol, propanol, isopropanol, butanol, and the like, or mono- and/or dialkylether solvents such as propyleneglycol monomethyl ether or ethylene glycol monophenyl or monobutyl ether, and the like, to these compositions. Additionally, non-VOV solvents, such as acetone, methyl acetate, d-limonene and the like may find use in the present invention. The water in the composition may be present from about 1% to about 90%, the VOC-contributing solvents at less than 2%, and the non-VOC contributing solvents or co-solvents may be present from about 1% to about 80% by weight. Some ethanol may still be present in the final compositions due to incorporation through some of the raw materials, but it is preferred to eliminate most of the ethanol from the polymeric raw materials and not to purposely add any additional VOC contributing solvents unless the total VOC of the composition can still be kept below 2%.

Exemplary embodiments of hair-hold compositions of the present invention are shown below in TABLE 1 as variants of Formula #5. Also in the table are Formulas #1-4 that did not show the hair-hold and non-flaking characteristics necessary for a marketable product. Formula #5 below shows remarkable hard-hold characteristics because it includes the requisite polyol, in this example sorbitol or mannitol or combinations of the two. That is, Formula #5 was made up with mannitol, with sorbitol, and with combinations of both and products with similar characteristics resulted. The physical properties of these formulas are also shown in the table.

TABLE 1

| | Formula 1 | Formula 2 | Formula 3 | Formula 4 | Formula 5 |
|---|---|---|---|---|---|
| Ingredients | | | | | |
| Chelant | 0.05 | 0.0 | 0.0 | 0.0 | 0.0 |
| Hydroxyethylcellulose | 0.30 | 0.30 | 0.30 | 0.30 | 0.30 |
| Amaze ® (National Starch, modified corn starch) | 0.50 | 0.50 | 0.50 | 0.50 | 0.50 |
| Petrolatum or other waxes and oils⁶ | 2.0⁶ | 1.0⁷ | 2.0⁶ | 2.0⁶ | 2.0⁸ |
| Fatty alcohols¹ | 1.5 | 1.5 | 1.5 | 1.5 | 1.5 |
| Emulsifiers³ | 2.25 | 2.25 | 2.25 | 2.25 | 1.05 |
| Oil soluble film former² | 1.5 | 1.5 | 1.5 | 1.5 | 1.5 |
| Polyvinylpyrrolidone | 20.0 | 25.0 | 20.0 | 20.0 | 20.0 |
| Polyvinylpyrrolidone/vinyl acetate copolymer | 10.0 | 10.0 | 10.0 | 10.0 | 10.0 |
| Vinyl pyrrolidone/dimethylaminoethyl | 10.0 | 11.8 | 10.0 | 10.0 | 10.0 |
| methacrylate copolymer | | | | | |
| Vinyl caprolactam/vinyl | 10.0 | 12.0 | 12.0 | 12.0 | 12.0 |
| pyrrolidone/dimethylaminoethyl methacrylate | | | | | |
| copolymer⁴ | | | | | |
| Polyol (sorbitol or mannitol) | 0.0 | 0.0 | 0.0 | 0.0 | 10.0 |
| Adjuvant(s) | q.s. | q.s. | q.s. | q.s. | q.s. |
| Solvents/co-solvents⁵ | 0.0 | 0.0 | 4.0 | 6.0 | 0.0 |
| Water | q.s | q.s. | q.s. | q.s. | q.s. |

PHYSICAL PROPERTIES

| | | | | | |
|---|---|---|---|---|---|
| Appearance | Opaque | Opaque | Opaque | Opaque | Opaque |
| | cream | cream | cream | cream | cream |
| Color | Off-white | Off-white | Off-white | Off-white | Off-white |
| Odor | Floral | Floral | Floral | Floral | Floral |
| pH | 6.54 | 5.78 | 5.81 | 5.73 | 5.47 |
| Viscosity (#TE@10 rpm) | 160,000 | 100,000 | 100,000 | 125,000 | 175,000 |
| 1-Mo Stability | Slightly | Slightly | Separated | Good | Good |
| | separated | separated | | | |
| VOC (calculated) | 6.3% | 2% | 2% | 2% | 0.1% |

¹Formula #5 included a 1:1 blend of cetyl and stearyl alcohols.
²Formula #5 included Ganex ® WWP-660.
³Formula #5 included 0.75% Brij ® 721 and 0.30 Apifil combined.
⁴Formula #1 included 10.0% Advantage ® LC-A, Formula #2 used a mixture of 9.0% Advantage ® S and 3.0% Advantage ® LC-A (that differ in the solvent content), Formulas #3-5 each used 12.0% Advantage ® S.
⁵For Formulas #1-2, the solvents are not purposely added but there is ethanol present from the use of ethanolic solutions of fixative polymers as mentioned in footnote 4, Formula #3 had purposely added 2.0% denatured SDA alcohol and 2.0% methyl acetate solvents, Formula #4 had purposely added 2.0% denatured SDA alcohol and 4.0% acetone, Formula #5 had no purposely added solvents but a very small amount of ethanol will still be present from use of the Advantage ® S that h
⁶2.0% petrolatum.
⁷Formula #2 was 1.0% of C₁₂-C₁₆ benzoic acid fatty ester blend.
⁸Formula #5 may incorporate 2.0% petrolatum and/or Polawax (interchangeable or combined).

Referring to the compositions represented in TABLE 1 as Formulas #1-#5, Formula #1 is considered a control formula and is a good representation of the current state of the art with 6.3% VOC. It is the benchmark for performance as it provides excellent stiff hold but does not flake due to brittleness when combed. However, the relatively high VOC content makes it untenable for the market in view of VOC regulations. Also, consumers respond favorably to its viscous, glue-like rheology and the feel when in use, and thus any proposed alternatives to this state of the art benchmark formula must meet these standards of consumer acceptance and performance.

Formula #2 was an attempt to solve the VOC issue while trying to maintain stiff non-brittle hold by using a fatty acid ester in place of any solvents. Although Formula #2 achieves the desired <2% VOC, it has less hold and showed flaking on hair when combed, as compared to the benchmark Formula #1.

Formula #3 was an attempt to solve the VOC problem as well, but replacing any VOC contributors with non-VOC methyl acetate. Formula #3 also has about 2% VOC, but it showed less hold compared to Formula #1 and was phase unstable.

Formula #4 was an attempt to solve the VOC issue by using acetone as a non-VOC co-solvent. Formula #4 was unacceptable for similar reasons as Formula #3.

Formula #5 as mentioned is an example of a preferred embodiment of the present invention where sorbitol (or equivalently, mannitol) is used to stiffen the hold of an aqueous polymeric hair styling composition. Formula #5 provides hard-hold characteristics without altering the feel and appearance of the hair. Formula #5 also has the advantage of having virtually no VOC. Formula #5 may be made up with sorbitol or mannitol or both, and may include petrolatum or Polawax or both waxes.

TABLE 2 below summarizes a consumer test in six homes with each of the consumer testers comparing Formula #5 versus Formula #1 in a blind test:

TABLE 2

| | Formula | |
|---|---|---|
| Participant | Preferred | Open Comments |
| 1 | #1 | Feel, appearance and spread was better with #1 |
| 2 | #5 | #5 had overall better smell, texture and hold |
| 3 | #5 | #5 had better appearance, hold, dry time, and |
| | | overall performance. |
| 4 | #5 | #5 had likable hold and feel, and it was hard to |
| | | maintain spiked hair with #1. |
| 5 | #5 | #5 gave hair that did not get frizzy |
| 6 | #5 | Participant preferred #5 |

In summary, we have invented unique hair hold compositions that rely on the incorporation of a polyol such as sorbitol or mannitol to achieve hard-hold characteristics without undue brittleness and flaking. In this way, very low to no-VOC hair hold compositions are possible.

We claim:

1. A hair styling composition comprising;
a. from about 30% to about 70% of a synthetic polymer or copolymer;
b. from about 0.1% to about 5% of a cellulosic material;
c. from about 5% to about 15% of a polyol; and,
d. water,
wherein the final composition has a VOC content of less than 2%.

2. The composition of claim 1 wherein said synthetic polymer or copolymer is selected from the group consisting of polyvinylpyrrolidone, polyvinylpyrrolidone/vinylacetate copolymer, vinyl pyrrolidone/dimethylaminoethyl methacrylate copolymer, and vinyl caprolactam/vinyl pyrrolidone/dimethylaminoethyl methacrylate copolymer, and mixtures thereof.

3. The composition of claim 1 wherein said cellulosic material is selected from the group consisting of modified starch, and hydroethylcellulose, and mixtures thereof.

4. The composition of claim 1 wherein said polyol is selected from the group consisting of erythritol, glycerol, mannitol, sorbitol, xylitol, isomalt, lacitol, and maltitol, and mixtures thereof.

5. The composition of claim 4 wherein the said polyol is sorbitol or mannitol.

6. The composition of claim 4 further including an oil soluble film-forming polymer.

7. The composition of claim 4 further including a fatty alcohol.

8. The composition of claim 4 further including a wax or oil selected from the group consisting of petrolatum, Polawax, bees' wax, candelilla wax, carnauba wax, Jojoba oil, safflower oil, tribehenin, tallow, lard, palm oil, castor oil, sunflower seed oil, and soya bean oil, and mixtures thereof.

9. The composition of claim 4 further including fragrance, dye, pigment, and preservative, and mixtures thereof.

10. The composition of claim 4 further including an emulsifier.

11. A hair styling composition comprising;
a. from about 40% to about 60% of a synthetic polymer or copolymer selected from the group consisting of polyvinylpyrrolidone, polyvinylpyrrolidone/vinylacetate copolymer, vinyl pyrrolidone/dimethylaminoethyl methacrylate copolymer, and vinyl caprolactam/vinyl pyrrolidone/dimethylaminoethyl methacrylate copolymer, and mixtures thereof;
b. from about 0.1% to about 5% of a cellulosic material chosen from the group consisting of modified starch, and hydroxyethylcellulose, and mixtures thereof;
c. from about 5% to about 15% of a polyol;
d. from about 0.01% to about 5% of a fatty alcohol;
e. from about 0.1% to about 5% of a wax chosen from the group consisting of petrolatum, Polawax, bees' wax, candelilla wax, carnauba wax, Jojoba oil, safflower oil, tribehenin, tallow, lard, palm oil, castor oil, sunflower seed oil, and soya bean oil, and mixtures thereof; and,
f. water,
wherein the final composition has a VOC content of less than 2%.

12. A hair styling composition comprising;
a. from about 40% to about 60% of a synthetic polymer or copolymer selected from the group consisting of polyvinylpyrrolidone, polyvinylpyrrolidone/vinylacetate copolymer, vinyl pyrrolidone/dimethylaminoethyl methacrylate copolymer, and vinyl caprolactam/vinyl pyrrolidone/dimethylaminoethyl methacrylate copolymer, and mixtures thereof;
b. from about 0.1% to about 5% of a cellulosic material chosen from the group consisting of modified starch, and hydroxyethylcellulose, and mixtures thereof;
c. from about 5% to about 15% of a polyol;
d. from about 0.01% to about 5% of a fatty alcohol;
e. from about 0.1% to about 5% of a wax chosen from the group consisting of petrolatum, Polawax, bees' wax, candelilla wax, carnauba wax, Jojoba oil, safflower oil, tribehenin, tallow, lard, palm oil, castor oil, sunflower seed oil, and soya bean oil, and mixtures thereof; and,
f. water,
wherein the final composition has a VOC content of less than 1%.

13. A hair styling composition comprising;
a. from about 40% to about 60% of a synthetic polymer or copolymer selected from the group consisting of polyvinylpyrrolidone, polyvinylpyrrolidone/vinylacetate copolymer, vinyl pyrrolidone/dimethylaminoethyl methacrylate copolymer, and vinyl caprolactam/vinyl pyrrolidone/dimethylaminoethyl methacrylate copolymer, and mixtures thereof;
b. from about 0.1% to about 5% of a cellulosic material chosen from the group consisting of modified starch, and hydroxyethylcellulose, and mixtures thereof;
c. from about 5% to about 15% of sorbitol or mannitol or mixtures thereof;
d. from about 0.01% to about 5% of a fatty alcohol;
e. from about 0.1% to about 5% of a wax chosen from the group consisting of petrolatum, Polawax, bees' wax, candelilla wax, carnauba wax, Jojoba oil, safflower oil, tribehenin, tallow, lard, palm oil, castor oil, sunflower seed oil, and soya bean oil, and mixtures thereof; and,
f. water,
wherein the final composition has a VOC content of less than 1%.
